(19) United States
(12) Patent Application Publication
(10) Pub. No.: US 2026/0060537 A1
(43) Pub. Date: Mar. 5, 2026

(54) DEVICE FOR DETERMINING THE LENGTH OF AN OBJECT, IN PARTICULAR THE LENGTH OF AN EYE

(71) Applicant: Heidelberg Engineering GmbH, Heidelberg (DE)

(72) Inventors: Frank Karlheinz MÜLLER, Speyer (DE); Tilman Philipp OTTO, Limburgerhof (DE)

(73) Assignee: Heidelberg Engineering GmbH, Heidelberg (DE)

(21) Appl. No.: 19/108,207

(22) PCT Filed: Jun. 28, 2023
(86) PCT No.: PCT/EP2023/067634

§ 371 Date: Mar. 3, 2025

(30) Foreign Application Priority Data
Sep. 1, 2022 (DE) 10 2022 122 164.4

(51) Int. Cl. A61B 3/10

(52) U.S. Cl. CPC A61B 3/1005; A61B 3/102

(57) ABSTRACT

The invention relates to a device (1) for performing optical coherence tomography (OCT), comprising: an interferometer (1a) for guiding a light beam (4) into a dispersive object to be examined, which object influences the propagation rate of light (4a, 4b, 4c) depending on the frequency thereof; and an evaluation unit (5) for detecting a length (3) of the object. With respect to the problem of detecting the length of a transilluminated object as reliably as possible by means of a device for performing optical coherence tomography, the device is characterised in that the evaluation unit (5) analyses interferometric data obtained from an OCT signal or interference spectrum and determines dispersion-related data of the interferometric data and determines the length (3) using the dispersion-related data.

The invention relates to a device according to the preamble of claim 1.

The term optical coherence tomography (typically abbreviated by OCT) is understood to mean an imaging method. Two-dimensional and three-dimensional images of light-scattering structures can be obtained using this method.

In this method, light having a certain bandwidth is typically split into two partial beams in a beam splitter. The first partial beam is incident on the sample or object to be examined, the second partial beam passes through a reference section.

The light reflected from the sample or the object interferes with the reference beam. By way of signals from the interference, the sample can be examined with depth resolution, thus in the depth of the optical axis of the first partial beam, by so-called A-scans.

In addition, it is also possible to scan the sample flatly or laterally using the first partial beam in order to obtain three-dimensional OCT images.

In so-called TD-OCT (Time Domain OCT), upon change of the optical path length in the reference arm, signals are continuously detected in the time domain, i.e. an intensity is detected as a function of time.

In so-called FD-OCT (Frequency Domain OCT), the interference of individual spectral components is detected, i.e. an intensity is detected as a function of the frequency or wavelength of the light.

In OCT imaging on a posterior segment of an eye, lateral scaling of the images is often only known as an image field angle. Information about the length of the eye and about the optical properties of the individual examined eye is necessary for converting the image field angle into absolute lengths or distances.

In addition, it is necessary to know the length of an eye if one wishes to perform a correction of an OCT image in order to display it in the correct scale having a correct curvature.

The dimensions of eyes and also visual defects vary significantly in the population. An A-scanned cSLO image or an OCT section is in different sizes without correction.

Against this background, presently two possibilities are known above all in order to determine the length of an eye.

An estimation of the length can be performed by a set refraction and a manually input radius of the cornea with the aid of an eye model.

A direct measurement of the length can also be performed using a separate device. Against this background, biometric devices are known which simultaneously record a rough image of the retina, for which a scaling can be determined using the length determined by the devices.

However, these images are not suitable for diagnostic purposes due to their poor quality.

A separate measurement of the length of an eye or the curvature of the cornea (corneal curvature) is an additional effort for a user. These measurements are therefore not always performed or the results are not transferred into software for assessing OCT images of the posterior eye segment.

If such values are entered and the entry takes place manually, transfer errors can additionally occur. The additional installation of the technology of a biometer in a diagnostic retina OCT device would significantly increase a system complexity.

The invention is therefore based on the object of detecting the length of a transilluminated object as reliably as possible by means of a device for performing optical coherence tomography.

The present invention achieves the above-mentioned object by way of the features of claim 1.

It has first been recognized according to the invention that light which penetrates into a dispersive object to be examined experiences a dispersion, namely that the object influences the propagation speed of the light as a function of its frequency. Furthermore, it has been recognized that an evaluation unit has to be provided in such a device, which analyzes the interferometric data obtained from an OCT signal or interference spectrum, determines dispersion-related data of the interferometric data, and determines the length of the object by means of the dispersion-related data. According to the invention, the axial length of the object, in particular the human eye, is measured by detecting the dispersion. It has been recognized here according to the invention that dispersion is normally an interfering factor which results in worse image quality, because of which dispersive effects in OCT devices are compensated for via hardware and/or software. However, the effect of the dispersion is used according to the invention to measure lengths.

Using such a device, it is possible to estimate the length of an eye by evaluating OCT signals in a relatively problem-free manner. Furthermore, it is possible to measure the length of an eye during an OCT image capture on the posterior eye segment.

In existing devices, the corneal curvature and the refraction are used to estimate the axial length of the eye and thus determine a scaling. The determination of the axial length of the eye is flawed, however, if ocular parameters which are not considered, such as a corneal curvature of the second face, the anterior chamber depth, and lens parameters deviate from the model eye used. This is the case in particular if a refraction defect of a patient was corrected by using intraocular lenses (IOLs) or by refractive surgery. The direct measurement of the axial length of the eye is therefore a significantly more robust parameter for a scaling determination. Classification methods can achieve a higher test power due to the better correspondence of the scaling between test data and reference data.

Methods in which a dense volume is not recorded, but which use scanning patterns having fixed scaling additionally profit from better correspondence of the recording locations of the recorded OCT sectional images with the target positions. For example, with circular scans having a selected absolute radius, in particular in the millimeter range, the actual radius in the eye will vary less.

The evaluation unit could determine the length of the object by a fit to a model or on the basis of a model which represents the influence of dispersion on light as a function of the path length which the light has passed through in a dispersive medium. In transparent media, an index of refraction depends on the frequency of the light incident in these media. This effect of the dispersion is used for the determination of the length of the object.

The evaluation unit could make use of a predetermined model, which theoretically describes the dispersion, namely the influence of a medium on the propagation speed of light in this medium. Such models could be stored in a storage medium of the device. The best matching model can be used depending on the transilluminated object in order to approximately theoretically describe the dispersion behavior of the respective examined object and conclude the length of the object on the basis of experimental values.

The model could describe the dispersion behavior of one medium or multiple media of the human eye. In this way, measurements of the length of an eye are possible reliably. The naturally existing dispersion of ocular media can be used to determine the length of the eye when carrying out an FD-OCT, in particular without additional hardware.

The interferometric data could comprise A-scans or OCT images, which are generated as partial spectra of an interference spectrum. A-scans are generated in the depth of a beam direction, so that dispersion influences longitudinally on an optical path length can be concluded therefrom.

Against this background, the evaluation unit could determine an axial distance of every two A-scans or two OCT images along a beam direction to determine the dispersion-related data.

The evaluation unit could lay a matching curve or a fit curve through values which are obtained from the dispersion-related data in order to determine the length of the examined object. A length dimension can thus be concluded from experimentally determined data, wherein theoretical values are compared to experimental values.

The device used here could be designed as an FD-OCT, namely as a device which is suitable for performing frequency domain optical coherence tomography (FD-OCT), in particular for performing spectral domain optical coherence tomography (SD-OCT), or for performing swept source optical coherence tomography (SS-OCT).

A device of the type described here could be used in a method for determining the length of a human eye, wherein the method comprises the following steps:
recording an interference spectrum using the device;
dividing the interference spectrum into two or more partial spectra;
calculating an A-scan or an OCT image for each partial spectrum;
determining the relative axial offset of every two A-scans or OCT images in the optical path length in relation to one another in order to conclude the dispersion behavior of the eye;
determining the length of the eye on the basis of a model, in particular a matching curve or fit curve, which represents the influence of dispersion on light as a function of the path length which the light has passed through in a dispersive medium.

Using such a method, an estimation of the length of an eye by means of the use of frequency domain optical coherence tomography (FD-OCT) is implementable. The method comprises at least the following steps: The method comprises the step of recording FD-OCT data on the eye, the step of determining a dispersion behavior, preferably via a suitable method, and the step of calculating the axial length of the eye by means of a fit, in particular a fit curve or matching curve, based on a model of the dispersion behavior as a function of the length of the dispersive ocular media which are passed through.

Various methods are possible for the determination of the dispersion from an OCT signal: One suitable method is dispersion determination via a spectral axial shift, namely the so-called “walk-off shift” method, which consists of the following method steps: An interference spectrum is recorded using a spectral domain OCT. The spectrum is divided into two or more partial spectra. The calculation of an A-scan or OCT image is carried out for each individual one of these partial spectra. The relative axial offset in optical path length of the A-scans or OCT images of the various partial spectra in relation to one another is determined. A dispersion curve is determined by a model fit.

Multiple A-scans or OCT images could be used together, in particular strips of adjacent A-scans or OCT images, in order to determine the relative axial offset of OCT images from the partial spectra. The axial relative positions of the OCT images can thus be estimated from the partial spectra with higher accuracy. Decentralized A-scans or A-scans from various locations of the ocular fundus could also be calculated in order to determine the shape of the ocular fundus. In conjunction with an OCT device which scans the retina, the determination of the thickness of the medium passed through, in particular the length of the eye, can also be carried out using non-central A-scans and for each scan coordinate separately. Even more detailed information about the shape of the ocular fundus can thus be obtained.

An OCT signal of the retina could be recorded and the length of the eye could be determined and/or an OCT signal could be recorded for the posterior eye segment and the length of the eye could be determined. The length of the eye can thus be determined upon an examination of the ocular fundus, in particular the retina. Advantageously, a separate measurement of the length of the eye is not necessary, and no error source occurs in the transfer of data or due to the non-input of data. The average accuracy of an absolute scaling specification of retina OCT recordings, and of simultaneously recorded cSLO images, namely of images from confocal scanning laser ophthalmoscopy (cSLO), thus increases.

The length of the eye and the axial position of an OCT image could be used to measure and/or set the distance between a camera of the device and the eye. The user therefore has to perform fewer adjustment steps in order to examine an eye. The distance between the camera and the eye can be calculated from the calculated length of the eye together with the axial absolute position of the OCT image. If information about the position of the fovea is additionally available in the software, for example, from an anatomical positioning system, the central length of the eye can be determined with greater reliability.

The OCT signal of the posterior eye segment, in particular the retina, could be used as a direct control variable for an automatic setting of the distance of a camera of the device from the eye, after its length is determined. It can advantageously be checked during the recording by a distance measurement, calculated from the length of the eye and OCT position on the retina, whether the distance of the camera to the eye is correct. This information can be used for the manual or automatic adjustment of the camera. The OCT signal of the retina can be used as a direct control variable (retina signal in the sweet spot for reference arm length set optimally for eye length) for the automatic distance setting, if the optical length of the eye is known.

The curvature of the posterior eye segment, in particular the retina, could be determined. The method advantageously does not require any additional hardware beyond a FD-OCT system. A measurement of the curvature of the retina is relevant for diverse pathologies, for example, in myopia patients. The curvature of the retina signal within the OCT image field is essentially dependent on the working distance apex-objective/apex-cornea.

If the optical axial length is known, the working distance can be reliably determined from the known parameters reference arm length and sample arm length to the objective apex. The true curvature of the retina can thus be determined significantly more accurately using corresponding eye models. In principle, there is the advantage over methods which measure the distance to the retina and cornea sequentially, for example, in that the reference arm length is varied, that the measurement takes place simultaneously. Errors in the length measurement due to axial movement of the eye are thus largely or entirely precluded.

The teaching described here permits classification methods which depend on scaling to be made more accurate. The teaching offers aid in the manual adjustment by specifying the eye distance or derived indications, therefore on average a higher image quality is achieved. An automatic adjustment function of the device is assisted. There is the possibility of displaying the retina in the correct scale with actual curvature.

### IN THE FIGURES OF THE DRAWINGS

FIG. 1 shows a schematic representation of a recording of the posterior eye segment by means of OCT or cSLO, in which the field of view angle is known as a device parameter φ, but the size of the recorded area at the fundus d is not directly accessible, because the optics and size of the eye are not known,

FIG. 2 shows a schematic representation of different eyes, which each display different lengths,

FIG. 3 shows a diagram, which represents the index of refraction n on the y axis in relation to the wavelength of the light in μm on the x axis,

FIG. 4 shows a schematic representation of an eye in which three light beams having different frequencies have different wavelengths in the eye interior due to the dispersive properties of the ocular media,

FIG. 5 shows a schematic representation of a device for carrying out optical coherence tomography (OCT), which guides a light bundle into an eye to be examined, and an evaluation unit, which analyzes interferometric data obtained from an OCT signal or interference spectrum and determines dispersion-related data of the interferometric data and determines the length of the eye by means of the dispersion-related data,

FIG. 6 shows a schematic representation of a calculation of two OCT images from partial spectra of a spectrum,

FIG. 7 shows a schematic representation of the axial offset of the two registered OCT images from FIG. 6, and

FIG. 8 shows a fit curve, from which the eye length is determinable on the basis of the axial offset, wherein the fit curve comprises a graph in which the length of the eye in mm is plotted on the x axis and the axial offset is plotted on the y axis.

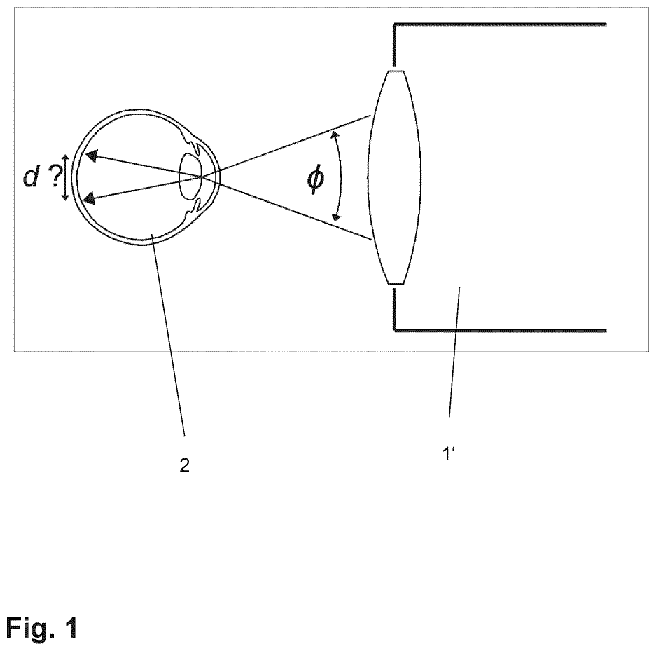

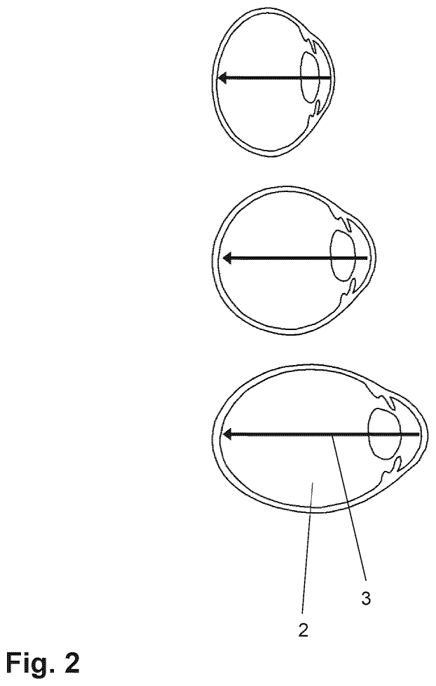

FIG. 1 shows a schematic representation of a recording of the posterior eye segment by means of a device 1′ for carrying out optical coherence tomography, in which the field of view angle is known as a device parameter φ. However, the size of the recorded area at the fundus d is not directly accessible, because the optics and size of the eye 2 are not known. FIG. 2 shows different eyes 2 having different lengths 3.

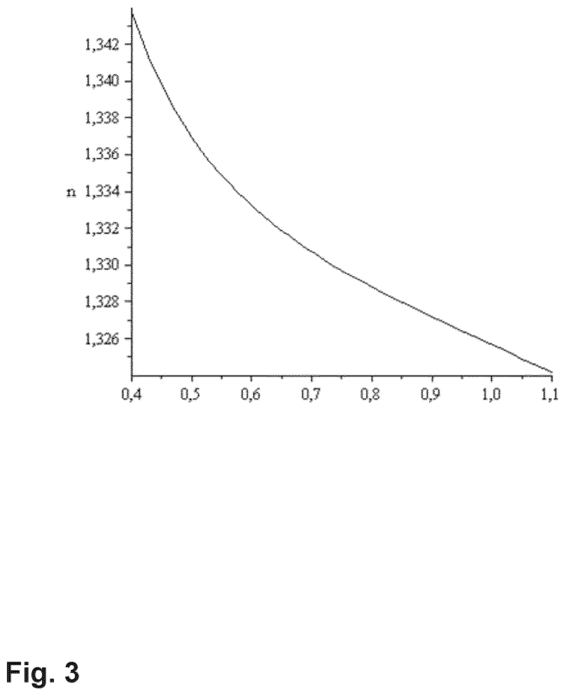

FIG. 3 shows mathematically that the propagation speed of light in dispersive media is dependent on its frequency. The index of refraction n of a medium is calculated from the quotient of the wavelength of the light in vacuum to the wavelength of the light in the material.

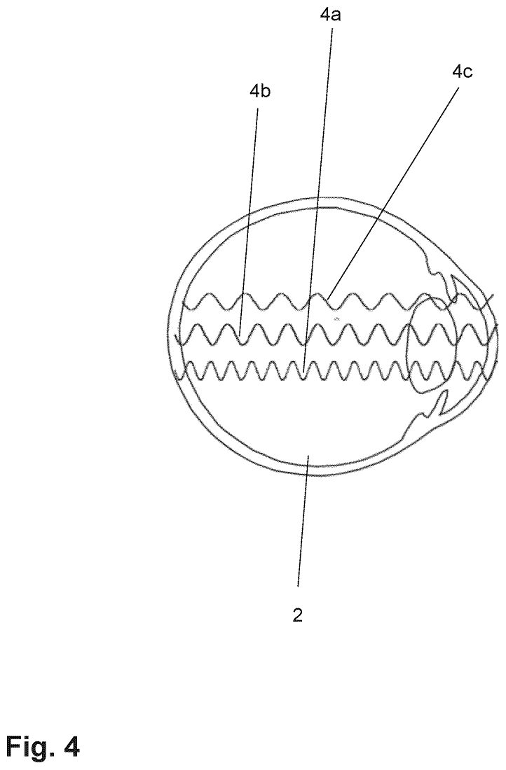

FIG. 4 shows schematically that three light beams having different frequencies have different wavelengths in the interior of the eye 2 and therefore have different propagation speeds. An optical path length is different for different wavelengths due to the dispersion with equal physical distance. The more dispersive the medium which the light passes through, the more the optical path lengths differ for different wavelengths. That is to say, the longer the eye 2 is, the stronger is the effect of the dispersion.

The length of the eye can be calculated from the strength of the dispersion effects using a model n (λ, z) for the dispersion of an eye. In an exemplary model for a homogeneously constructed eye, the following applies:

$$L ( λ ) = n ( λ ) \cdot d$$

In this formula, L is the optical path length for light of a specific wavelength λ, n is the index of refraction, and d is a physical distance through which light is to pass in a dispersive medium, thus the length of the modeled eye. The following then applies for the difference of optical path lengths L:

$$Δ L = L ( {λ}_{1} ) - L ( {λ}_{2} ) = ( n ( {λ}_{1} ) - n ( {λ}_{2} ) ) \cdot d$$

The length d of the modeled eye therefore results as:

$$d = \frac{Δ L}{n ( {λ}_{1} ) - n ( {λ}_{2} )}$$

An OCT device can be used to measure the ΔL, thus the difference of optical path lengths.

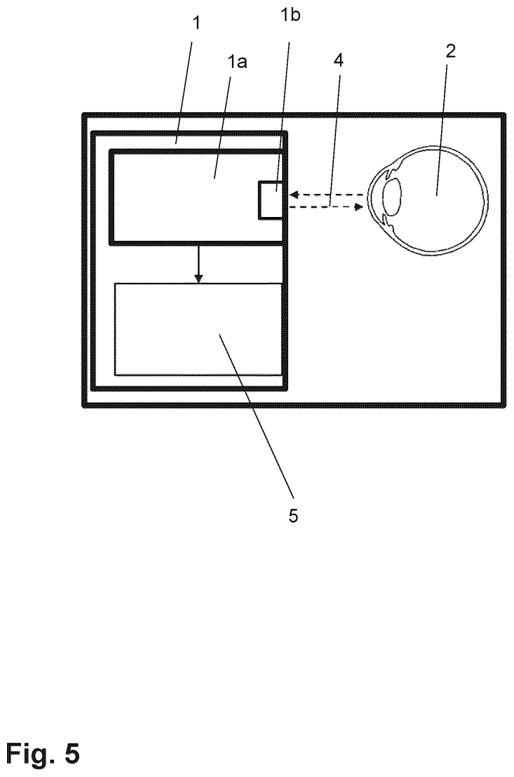

FIG. 5 shows such a device 1 for carrying out optical coherence tomography (OCT), comprising an interferometer 1a for guiding a light bundle 4 into a dispersive object to be examined, namely an eye 2, which influences the propagation speed of light as a function of its frequency, and an evaluation unit 5 for detecting a length 3 of the object, namely the eye 2.

The interferometer 1a splits light having a certain bandwidth into two partial beams in a beam splitter. The first partial beam is incident on the object to be examined, namely the eye 2, and the second partial beam passes through a reference section. The light reflected from the object interferes with the reference beam. By way of signals from the interference, the object can be examined with depth resolution, thus in the depth of the optical axis of the first partial beam, by so-called A-scans or OCT images.

The evaluation unit 5 analyzes interferometric data obtained from an OCT signal or interference spectrum and determines dispersion-related data of the interferometric data and determines the length 3 of the eye 2 by means of the dispersion-related data.

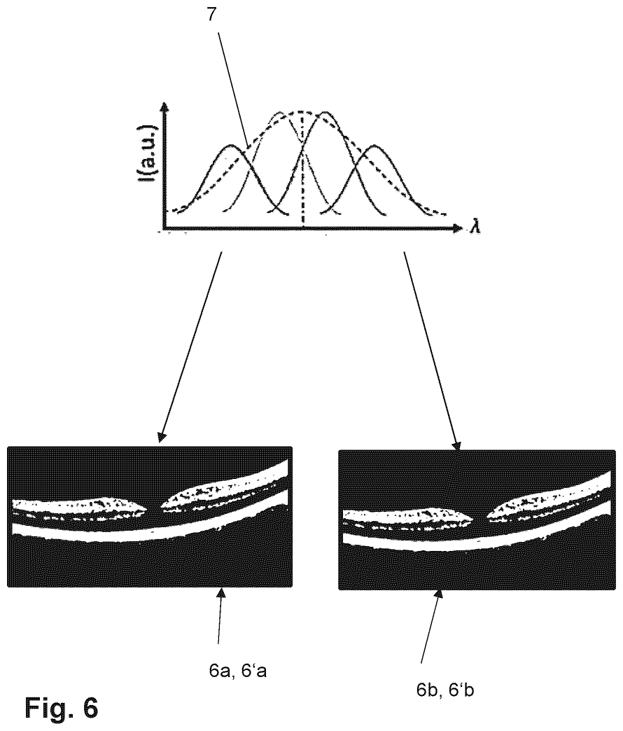

FIG. 6 shows on the basis of a so-called “split spectrum approach” that the interferometric data comprise A-scans 6a, 6b or OCT images 6′a, 6′b, which are generated or calculated from partial spectra of an interference spectrum 7. The OCT images 6′a, 6′b from the various partial spectra are registered.

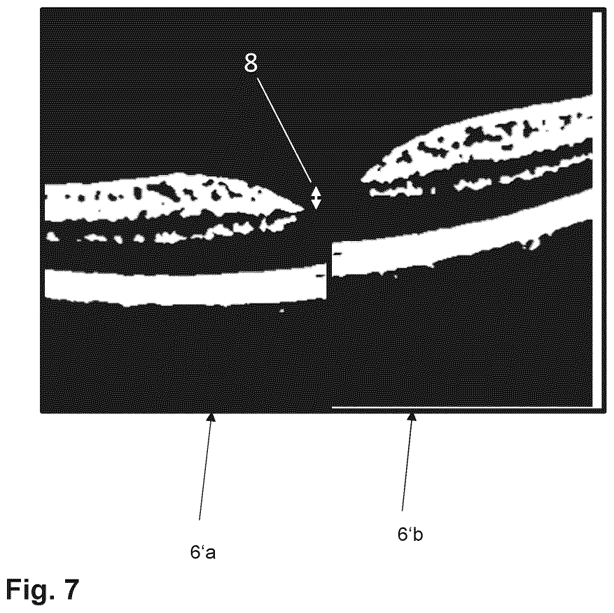

FIG. 7 shows that the evaluation unit 5 determines an axial distance 8 of the two A-scans 6a, 6b or the OCT images 6′a, 6′b along a beam direction to determine the dispersion-related data. Specifically, the dispersion-related data therefore comprise at least one axial offset 8 of the OCT images 6′a, 6′b.

The evaluation unit 5 uses multiple A-scans 6a, 6b or OCT images 6′a, 6′b together, namely specific strips of adjacent A-scans 6a, 6b or OCT images 6′a, 6′b, in order to determine the relative axial offset 8 of OCT images 6′a, 6′b from the partial spectra.

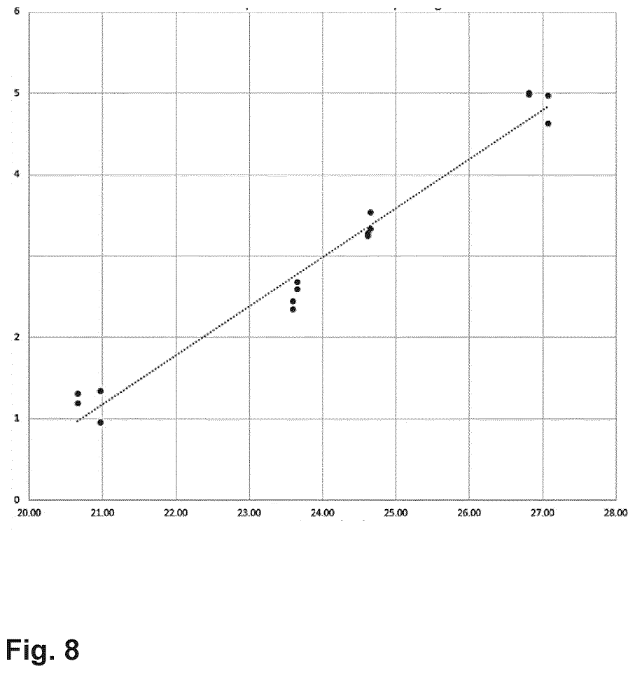

FIG. 8 shows that the evaluation unit 5 determines the length 3 of the eye 2 by way of a fit to a model or on the basis of a model which represents the influence of dispersion on light as a function of the path distance which the light has passed through in a dispersive medium.

Specifically, the evaluation unit 5 uses a predetermined model, which theoretically describes the dispersion, namely the influence of a medium on the propagation speed of light in this medium. The model according to FIG. 8 describes the dispersion behavior of one medium or multiple media of the human eye 2. The length 3 of the eye 2, thus the path distance which is passed through, can be read from the fit curve according to FIG. 8 on the x axis if the axial offset 8 is known.

The evaluation unit 5 lays a matching curve or fit curve through values, which are obtained from the dispersion-related data, in order to determine the length 3.

The device is designed as an FD-OCT, namely as a device which is suitable for carrying out frequency domain optical coherence tomography (FD-OCT), in particular for carrying out spectral domain optical coherence tomography (SD-OCT), or for carrying out swept source optical coherence tomography (SS-OCT).

Using the device described here, a method for determining the length 3 of a human eye 2 is carried out, which comprises the following steps:
Recording an interference spectrum 7 using the device 1;
dividing the interference spectrum 7 into two or more partial spectra;
calculating an A-scan 6a, 6b or OCT image 6′a, 6′b for each partial spectrum;
determining the relative axial offset 8 of every two A-scans 6a, 6b or OCT images 6′a, 6′b in the optical path length in relation to one another in order to conclude the dispersion behavior of the eye 2;
determining the length 3 of the eye 2 on the basis of a model, namely a matching curve or fit curve, which represents the influence of dispersion on light as a function of the path distance which the light 4 has passed through in a dispersive medium.

Multiple A-scans 6a, 6b or OCT images 6′a, 6′b are used together, in particular strips of adjacent A-scans 6a, 6b or OCT images 6′a, 6′b, in order to ascertain the relative axial offset 8 of images 6′a, 6′b from the partial spectra. Using the method, an OCT signal of the retina is recorded and the length 3 of the eye 2 is determined. An OCT signal is specifically recorded at the posterior eye segment and the length 3 of the eye 2 is determined.

The length 3 of the eye 2 and the axial position of an OCT image 6′a, 6′b can be used to measure and possibly set the distance between a camera 1b of the device 1 and the eye 2. The distance between camera 1b and eye 2 can be calculated from the length 3 of the eye 2 and the axial OCT position. The length 3 of the eye 2 can be measured at multiple arbitrary points of a B-scan.

The OCT signal of the posterior eye segment, in particular the retina, is used as a direct control variable for automatic setting of the distance of a camera 1b of the device 1 from the eye 2, after its length 3 is determined. The determined distance to the eye 2 is used to adjust the camera 1b during a recording. In particular, fully automatic adjustment of the camera 1b is assisted.

The curvature of the posterior eye segment, in particular the retina, is determined. The length 3 of the eye 2 can be used to display the retina in the correct scale with natural curvature.

### LIST OF REFERENCE SIGNS

1 device
1a interferometer of 1
1b camera of 1
2 eye
3 length of 2
4 light bundle
4a-c light, light beam having specific wavelengths
5 evaluation unit
6a, 6b A-scan
6′a, 6′b OCT image
7 spectrum
8 axial offset

1. A device for performing optical coherence tomography (OCT), comprising an interferometer for guiding a light bundle into a dispersive object to be examined, which influences the propagation speed of light as a function of its frequency, and an evaluation unit for detecting a length of the object,
characterized in that the evaluation unit analyzes interferometric data obtained from an OCT signal or interference spectrum and determines dispersion-related data of the interferometric data and determines the length by means of the dispersion-related data.

2. The device as claimed in claim 1, wherein the evaluation unit determines the length of the object by a fit to a model or on the basis of a model, which represents the influence of dispersion on light as a function of the path distance which the light has passed through in a dispersive medium.

3. The device as claimed in claim 1, wherein the evaluation unit makes use of a predetermined model, which theoretically describes the dispersion, namely the influence of a medium on the propagation speed of light in this medium.

4. The device as claimed in claim 2, wherein the model describes the dispersion behavior of one medium or multiple media of the human eye.

5. The device as claimed in claim 1, wherein the interferometric data comprise A-scans or OCT images, which are generated from partial spectra of an interference spectrum.

6. The device as claimed in claim 5, wherein the evaluation unit determines an axial distance of every two A-scans or every two OCT images along a beam direction to determine the dispersion-related data.

7. The device as claimed in claim 6, wherein the evaluation unit lays a matching curve or fit curve through values, which are obtained from the dispersion-related data, in order to determine the length.

8. The device as claimed in claim 1, characterized by a design as a FD-OCT, namely as a device which is suitable for performing frequency domain optical coherence tomography (FD-OCT), in particular spectral domain optical coherence tomography (SD-OCT) or swept source optical coherence tomography (SS-OCT).

9. A method for determining the length of a human eye using a device as claimed in claim 1, comprising the following steps:
recording an interference spectrum using the device;
dividing the interference spectrum into two or more partial spectra;
calculating an A-scan or an OCT image for each partial spectrum;
determining the relative axial offset of every two A-scans or every two OCT images in optical path length in relation to one another, in order to conclude the dispersion behavior of the eye;
determining the length of the eye on the basis of a model, in particular a matching curve or fit curve, which represents the influence of dispersion on light as a function of the path distance which the light has passed through in a dispersive medium.

10. The method as claimed in claim 9, wherein multiple A-scans or OCT images are used together, in particular strips of adjacent A-scans or OCT images, in order to determine the relative axial offset of images from the partial spectra.

11. The method as claimed in claim 9, wherein decentralized A-scans or A-scans from various locations of the ocular fundus are calculated to determine the shape of the ocular fundus.

12. The method as claimed in claim 10, wherein an OCT signal of the retina is recorded and the length of the eye is determined, and/or an OCT signal is recorded for the posterior eye segment and the length of the eye is determined.

13. The method as claimed in claim 9, wherein the length of the eye and the axial position of an OCT image are used to measure and/or set the distance between a camera of the device and the eye.

14. The method as claimed in claim 9, wherein the OCT signal of the posterior eye segment, in particular the retina, is determined as a direct control variable for automatic setting of the distance of a camera of the device from the eye, after its length is determined.

15. The method as claimed in claim 9, wherein the curvature of the posterior eye segment, in particular the retina, is determined.
